(19) United States
(12) Patent Application Publication
(10) Pub. No.: US 2024/0131247 A1
(43) Pub. Date: Apr. 25, 2024

(54) Arrangements for sterile fat harvesting

(71) Applicant: Medical JS Innovations Oy, Helsinki (FI)

(72) Inventor: Karl Johan Svedjeholm, Helsinki (FI)

(21) Appl. No.: 18/277,593

(22) PCT Filed: Feb. 17, 2022
(86) PCT No.: PCT/FI2022/050103

§ 371 Date: Aug. 16, 2023

(30) Foreign Application Priority Data
Feb. 17, 2021 (FI) 20215165

(51) Int. Cl. A61M 1/00; A61M 5/31

(52) U.S. Cl. CPC A61M 1/895; A61M 1/67; A61M 1/815; A61M 5/3135; A61M 2202/08; A61M 2209/045; A61M 2209/06

(57) ABSTRACT

According to an example aspect of the present invention, there is provided a sterile connector for fat harvesting, comprising a body configured to be at least partially inserted into a receptive syringe chamber. The body comprises an external interface end for facing the ambient space and an internal interface end for insertion into the syringe chamber. The connector further comprises a fat input conduit extending through the body. The fat input conduit comprises a coupling for a harvesting tube at the external interface end of the body and a feed port at the internal interface end of the body for feeding the harvested fat into the syringe chamber. The connecter further comprises an air output conduit extending through the body. The air output conduit comprises a coupling for a suction tube at the external interface end of the body and a suction port at the internal interface end of the body for providing a partial vacuum into the syringe chamber.

### FIELD

The present invention relates to a fat harvesting system. In particular, the invention relates to a connector in a fat harvesting system and a method for harvesting fat from a cannula using the system.

### BACKGROUND

Fat harvesting, or liposuction, refers to the procedure of removing fat from a human body. Fat transfer refers to the procedure where fat is harvested from one area of the body, purified and replaced into an another area of the body. Fat transfer can be used for surgical procedures like breast augmentation, facial fat transfer, lipofilling of scars or reconstruction of tissue with fat after cancer, tissue trauma, asymmetrical correction, wound healing etc. Especially in fat transfer the pressure in which the fat is harvested from the human body is critical. Fat cells are fragile and break if the vacuum effect or under pressure is too strong. If the vacuum effect or under pressure is too low, the procedure takes unnecessarily long.

Traditionally fat harvesting is performed in such a way that the harvested fat is sucked into a large container. This system works well when only a liposuction operation is conducted. However, if the fat is transferred back into the body, purifying the collected fat calls in the large container is difficult and even in some cases, not very sterile. Also, this method causes a lot of trauma to fragile fat cells.

One way for performing fat transfer is for the surgeon to operate a needle attached to a syringe by hand. This requires the surgeon to control the suction pressure by using the syringe by one hand and simultaneously using the harvesting needle to collect fat from the fat tissue. This technique is technically very difficult for the surgeon and impossible to control a recommended and stable suction pressure and causes uneven quality of the fat cells.

Different solutions for collecting and processing the fat have been introduced over the years. For example, patent application publication WO 2014098909 A1 and patent publication U.S. Pat. No. 8,172,832 B1 introduce a more sterile fat harvesting container. Patent publication U.S. Pat. No. 8,858,518 B2 introduces a canister and a method for processing the harvested fat.

### SUMMARY OF THE INVENTION

The aim of the invention is to provide an easy, fast and sterile way to perform fat harvesting and transfer. In particular, the invention aims to provide a fast and easy method for preforming fat transfer with very small and also larger amounts of fat and keeping the fat cells unbroken during the whole fat transfer process.

The invention is defined by the features of the independent claims. Some specific embodiments are defined in the dependent claims.

According to a first aspect of the present invention, there is provided a sterile connector for fat harvesting, comprising a body configured to be at least partially inserted into a receptive syringe chamber. The body comprises an external interface end for facing the ambient space and an internal interface end for insertion into the syringe chamber. The connector further comprises a fat input conduit extending through the body. The fat input conduit comprises a coupling for a harvesting tube at the external interface end of the body and a feed port at the internal interface end of the body for feeding the harvested fat into the syringe chamber. The connecter further comprises an air output conduit extending through the body. The air output conduit comprises a coupling for a suction tube at the external interface end of the body and a suction port at the internal interface end of the body for providing a partial vacuum (underpressure) into the syringe chamber.

According to a second aspect of the present invention, there is provided a sterile packaging including a connector, a first syringe, a harvesting tube and a plug, wherein the connector is hermetically sealed to the first syringe, the harvesting tube is connected to the coupling for a harvesting tube, and the plug is connected to the first syringe.

According to a third aspect of the present invention, there is provided a method for harvesting fat from a cannula, the method comprises the steps of providing a connector hermetically sealed to a first syringe, a harvesting tube connected to the coupling for a harvesting tube of the connector, a second syringe connected to the first syringe and a holder for supporting the first syringe, connecting the free end of the harvesting tube to a cannula, connecting the free end of the suction tube to a suction machine, and providing a partial vacuum by the suction machine. This way the vacuum effect or underpressure remains stable and easily adjustable by providing the vacuum effect to the desired level from the suction machine.

According to the fourth aspect of the present invention there is provided a fat harvesting set in a sterile packaging which includes a connector, a first syringe, a harvesting tube and at least one of a plug and second syringe with a luerlock connector.

Several advantages are achieved by the current invention. The current invention is helpful when very small, medium or bigger amounts of fat to be harvested from the body, treated and injected back into different parts of the body. The method according to the current invention is completely sterile and accurate. Also, the current invention is gentle for the fat cells to achieve a good outcome in fat transfer. The harvested fat can be utilized for injection without any further processing, in other words, the harvested fat is a ready product.

The current invention makes fat harvesting or transfer fast and easy as the connector according to one aspect of the current invention connects to conventional appliances, such as a standard syringe, in a simple way with minimal possibility of misuse. When using a standard transparent syringe with a scale the amount and the quality of the collected fat can be exactly measured in ml/cc and thus monitored easily. Also, the approximate time consumption to harvest 20-50 ml fat will be around 1-2 minutes.

### BRIEF DESCRIPTION OF THE DRAWINGS

FIG. 1 illustrates a connector in accordance with one embodiment of the present invention;

FIG. 2 illustrates a connector in accordance with the same embodiment as FIG. 1 seen from the opposite direction;

FIG. 3 illustrates side view of a connector of FIG. 1;

FIG. 4 illustrates side view of a connector according to another embodiment of the present invention;

FIG. 5 illustrates a side view of a fat harvesting system in accordance with a third embodiment of the present invention;

FIG. 6 illustrates a connector in accordance with one embodiment of the present invention;

FIG. 7 illustrates a connector in accordance with the same embodiment as FIG. 6 from a side view; and

FIG. 8 illustrates a holder for the fat harvesting system in accordance with an embodiment of the present invention.

### EMBODIMENTS

### Definitions

In the present context, the term “sterile” refers to a product that has been sterilized according to required medical standards so that all microorganisms have been removed or killed from the surface of the product. This means that the product is totally clean and free from all kind of microorganisms/microbes.

In the present context, the term “hermetic seal” refers to a sufficiently airtight seal which prevents the passage of air during the use of the fat harvesting system.

In the present context, the term “external interface” refers to the interface of the connector which is outside of the syringe chamber when the connector is in use, such as in attached to the syringe. Similarly, the term “external section” refers to the section of the connector which is outside of the syringe chamber when the connector is in use, such as in attached to the syringe.

In the present context, the term “internal interface” refers to the interface of the connector which is inside of the syringe chamber when the connector is in use, such as in attached to the syringe. Similarly, the term “internal section” refers to the section of the connector which is inside of the syringe chamber when the connector is in use, such as in attached to the syringe.

In the present context, the term “partial vacuum” refers to the pressure difference between the inside of the syringe and atmosphere, caused by a suction machine

As appears from above, it has been found that by adding a separate connector to an ordinary syringe the fat harvesting process can be simplified.

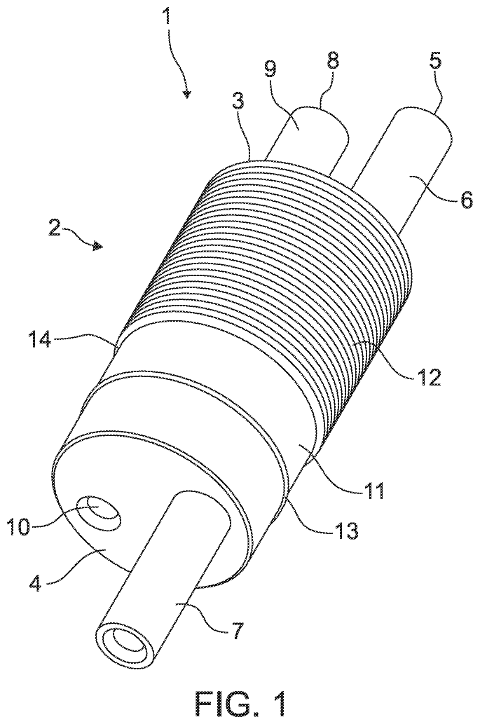

FIG. 1 illustrates a connector 1 in accordance with at least some embodiments of the present invention. The connector comprises a body 2 configured to be at least partially inserted into a receptive syringe chamber. The body 2 comprises an external interface end 3 for facing the ambient space and an internal interface end 4 for insertion into the syringe chamber. The connector 1 further comprises a fat input conduit 5 extending through the body that comprises a coupling for a harvesting tube 6 at the external interface end 3 of the body 2 and a feed port 7 at the internal interface end 4 of the body 2 for feeding the harvested fat into the syringe chamber. The connector 1 further comprises an air output conduit 8 extending through the body 2 that comprises a coupling for a suction tube 9 at the external interface 3 end of the body 2 and a suction port 10 at the internal interface end 4 of the body 2 for providing a partial vacuum into the syringe chamber.

According to one embodiment, the connector 1 is formed from one solid piece with no moving parts. This improves the safety of the connector, ensures that the harvesting process stays sterile, improves the patient safety as there is no risk of particles being transferred into the harvested fat and therefore to the patient. Also, without moving parts the partial vacuum stays constant which improves the quality of the harvested fat.

According to one embodiment, the coupling for the suction tube 9 and the coupling for the harvesting tube 6 extend out from the external interface end 3 of the body 2. The coupling for the suction tube 9 and the coupling for the harvesting tube 6 extend at least 15 mm, for example 15-25 mm, out from the external interface end 3 of the body 2. The coupling for the suction tube 9 and the coupling for the harvesting tube 6 do not have to extend out of the external interface end 3 of the body 2 the same amount, in other words their lengths can be either the same or different.

According to one embodiment the the body 2 comprises an internal section 11 housing the internal interface end 4, which internal section 11 is configured to be inserted into a receptive syringe chamber, and an external section 12 housing the external interface end 3, which external section 12 is configured to stay out of the receptive syringe chamber.

The lengths and diameters of the internal and external sections 11, 12 can vary. In one embodiment the internal section 11 is so long that it will fit into the receptive syringe, for example at least 1 cm long, for example at least 1.7 cm long, such as 2-3 cm long. Also, the diameter of the internal section 11 varies depending on the receptive syringe used. In one embodiment the diameter of the internal section 11 is be similar to the diameter of the chamber of the receptive syringe so the it will fit into it tightly. The length of the external section 12 may vary more than the length of the internal section 11. The length of the external section 12 can be at least 0.5 cm, for example at least 1.5 cm, such as 1.5-3.0 cm, or at least 20 mm. The diameter of the external section 12 can be the same, larger or smaller than the diameter of the internal section 11. According to one embodiment the diameter of the external section 12 is for example at least 2 mm larger than the diameter of the internal section 11. If the diameter of the external section 12 is smaller than the internal section 11, according to one embodiment, there is a shoulder 14 to prevent the external interface 3 from being inserted into the receptive syringe.

According to an embodiment, the diameter of the internal section 11 is 26.5-27.5 mm, preferably 27.37±0.1 mm so that it fits tightly into the syringe chamber of a standard 50 ml syringe.

According to an embodiment, the diameter of the internal section 11 is 14-15.5 mm, preferably 15±0.1 mm so that it fits tightly into the syringe chamber of a standard 20 ml syringe.

According to one embodiment the internal section 11 is adapted to form a hermetic seal when inserted into a receptive syringe chamber. The seal is formed by the surface shape of the internal section, which conforms to the shape of the internal surface of the syringe chamber and fits tightly in the syringe chamber. The seal also locks the connector to the receptive syringe. The standard syringe has an inner rim which helps lock the seal tightly into place.

According to one embodiment the hermetic seal is formed by a sealing 13. The sealing is made of a polymer material, such as thermoplastic elastomer (TPE).

According to one embodiment the sealing 13 is integrated into the body 2 of the connector 1. In other words, the sealing 13 cannot be removed from the body 2.

According to one embodiment the external section 12 has a grip surface. The grip surface is formed by an uneven or rough surface. The purpose of the grip surface is to help remove the connector 1 from the first syringe.

According to one embodiment when inserted into the syringe chamber the fat input conduit 5 extends deeper into the syringe chamber than the air output conduit 8. More specifically, the fat input conduit 5 extends 10-25 mm, or at least 10 mm, such as at least 15 mm, for example at least 20 mm deeper into the syringe chamber than the air output conduit 8. In an embodiment, the fat input conduit 5 extends close to the other end of the syringe chamber, for example as far as 1-3 mm from the bottom of the syringe chamber. In the connector of FIG. 1, this distance also corresponds to the length of the feed port 7, which is thus 10-25 mm, or at least 10 mm, such as at least 15 mm, for example at least 20 mm from the surface of the interface end 4.

According to one embodiment the fat input conduit 5 and the air output conduit 8 have an inner diameter. The inner diameter is large enough not to damage the fat cells. The inner diameter is greater than the collecting hole of the cannula that is used to harvest the fat from the patient. The inner diameter is for example 3-7 mm, such as about 5 mm. When the fat cells move from a smaller diameter surrounding to a larger diameter surrounding, the risk of damaging the fat cells is minimized.

According to one embodiment the body 2 has a shoulder 14 for preventing the external interface end 3 from being inserted into the receptive syringe chamber. In particular, the circumference of the external section 12 is larger than the circumference of the internal section 11 so that the interface of these sections forms a shoulder 14. Also, the shoulder helps prevent the protective glove of the user to be caught between the connector and the receptive syringe. Therefore, it increases the patient safety.

The connector 1 is made of a material that can be sterilized, such as metal, glass or a polymer material. According to one embodiment the connector 1 is made of a polymer material, for example perfluoroalkoxy (PFA), polyimide (PI), polyphenylene sulfone (PPSU), polypropylene (PP), polyether ether ketone (PEEK), polyoxymethylene (POM-C) or polyvinylidene fluoride (PVDF) or a mixture thereof.

According to an embodiment the connector is made of a biocompatible polymer material, such as biocompatible polypropylene.

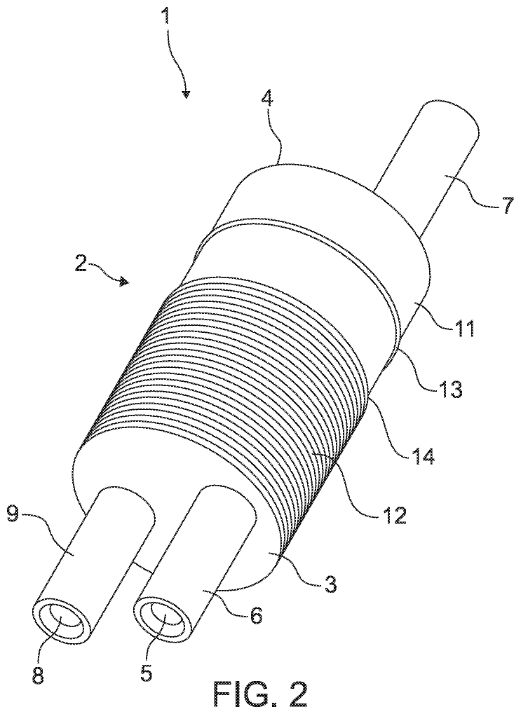

FIG. 2 illustrates a connector in accordance with the same embodiments as FIG. 1.

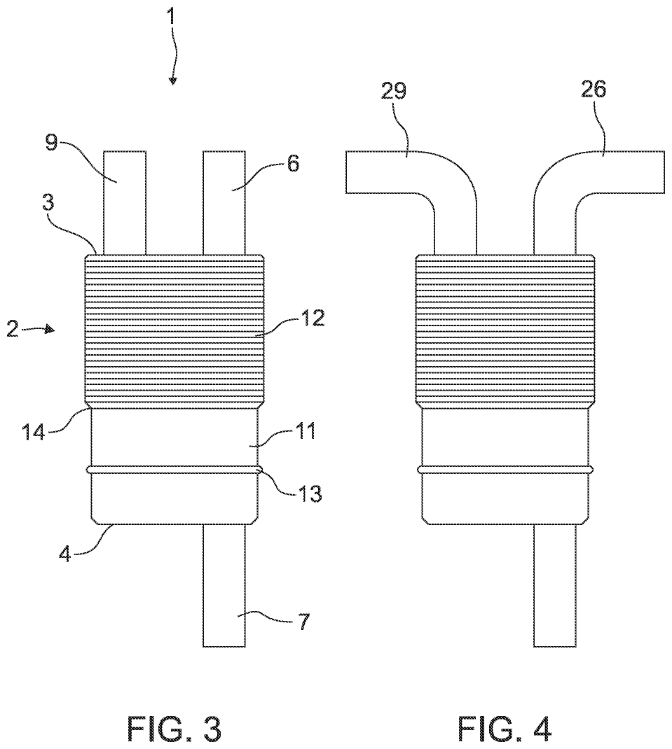

FIG. 3 illustrates side view of a connector in accordance with the same embodiments as FIGS. 1 and 2.

FIG. 4 illustrates a side view of a connector capable of supporting at least some embodiments of the present invention. According to this embodiment the coupling for the harvesting tube 26 and the coupling for the suction tube 29 are bent. The bend angle can be between 0° to 120°, such as 45 to 90°, or about 90°. The bend is rounded so that is does not have any sharp corners. This improves the quality of the collected fat cells as they will not collide with the sides of the tubes 26, 29 or other fat cells and break. In this embodiment the coupling for the harvesting tube 26 and the coupling for the suction tube 29 may be the same material as the connector 1 or some other material. The material can be flexible or expanding so that the harvesting tube and suction tube will fit tightly on the coupling for the harvesting tube 26 and the coupling for the suction tube 29. If the material of the coupling for the harvesting tube 26 and the coupling for the suction tube 29 is not flexible or expanding the harvesting tube and the suction tube may be fitted to the coupling for the harvesting tube 26 and the coupling for the suction tube 29 by an intermediate tube.

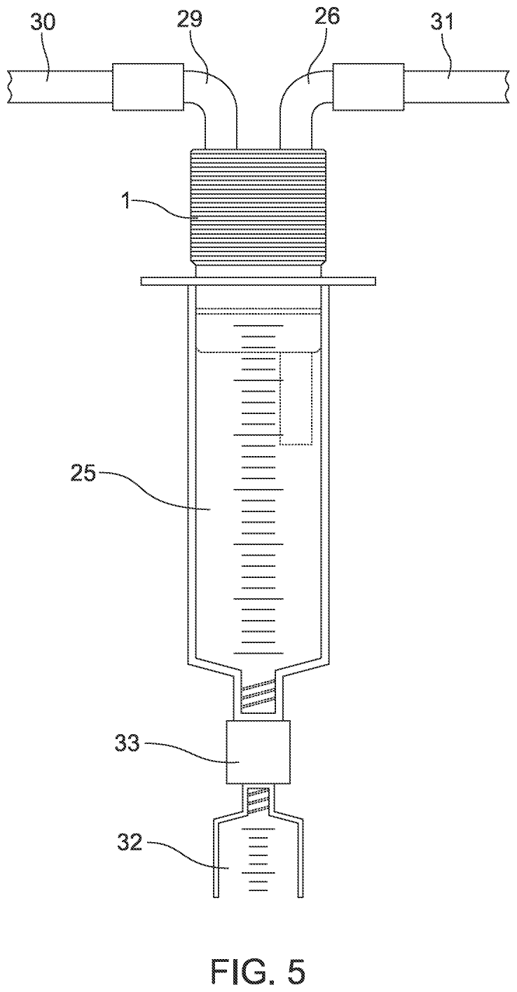

FIG. 5 illustrates a side view of a fat harvesting system in accordance with at least some embodiments of the present invention. The fat harvesting system includes includes a connector 1 according to any of the above mentioned embodiments, a first syringe 25, a harvesting tube 26 and at least one of a plug and a second syringe 32 with a luerlock 18, wherein the connector 1 is hermetically sealed to the first syringe 25, the harvesting tube 31 is connected to the coupling for a harvesting tube 26, and the plug or second syringe 32 with the luerlock 18 is connected to the first syringe 25.

According to one embodiment the first syringe 25 is a 50 ml luerlock syringe or a 20 ml luerlock syringe. Preferably, the first syringe is a standard syringe that is transparent and has a scale. A standard syringe is made of transparent plastic, typically polypropylene. It has a cylindrical syringe chamber for containing a fluid. It has two openings, one at both ends of the chamber. Otherwise it has an air tight structure. The air tight structure in addition with the standard syringe having a constant syringe chamber size, enables the partial vacuum to stay constant which improves the quality of the harvested fat.

According to one embodiment the fat harvesting system comprises at least one second syringe 32. This second syringe can be any size, but for example a 20 ml or 10 ml luerlock syringe is used.

According to one embodiment the second syringe 32 is connected to the first syringe 25 with a luerlock connector 18 replacing the plug. Therefore, the first syringe 25 is either connected to a plug or a second syringe 32 with a luerlock connector 18 at all times.

According to one embodiment the fat harvesting system comprises a suction tube 30 connected to the coupling for a suction tube 29.

According to one embodiment the fat harvesting system comprises a holder for supporting the first syringe. The holder can be made of any material. Preferably the holder is made of a polymer material or metal. The holder is described more precisely with reference to FIG. 8 below.

According to one embodiment a method for harvesting fat from a cannula is provided. The method comprises the steps of: providing the fat harvesting system according to the embodiments mentioned above, connecting the free end of the harvesting tube 31 to a cannula, connecting the free end of the suction tube 30 to a suction machine, and providing a partial vacuum by the vacuum machine.

According to one embodiment the provided partial vacuum is 1 bar or less, such as 0.2-0.8, for example 0.4-0.6. The pressure is indicated as a pressure difference between the ambient pressure and pressure provided by the vacuum machine. The pressure can be maintained constant due to the structure of the connector which does not have moving parts.

According to one embodiment there is provided a fat harvesting set in a sterile packaging which includes a connector 1 according to any of the above mentioned embodiments, a first syringe 25, a harvesting tube 26 and at least one of a) a plug and b) a second syringe 32 with a luerlock connector 18.

According to one embodiment the first syringe 25 is a 50 ml luerlock syringe or a 20 ml luerlock syringe.

According to one embodiment the fat harvesting set further comprises a suction tube 30.

According to one embodiment, the fat harvesting set further comprises a holder for supporting the first syringe 25.

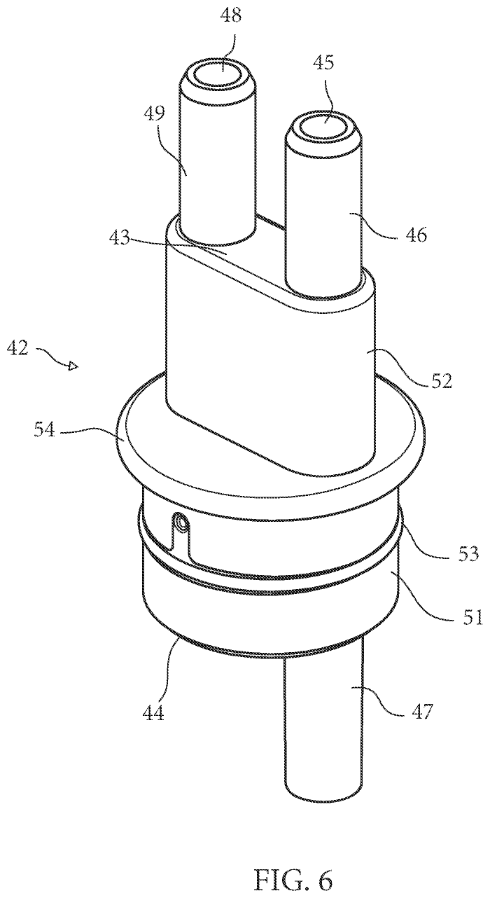

FIG. 6 illustrates a connector 1 in accordance with at least some embodiments of the present invention. The connector has a body 42, an internal section 51 and an external section 52. The internal section 51 of the connector is similar to embodiments explained above in accordance with FIGS. 1-4. The internal section comprises an internal interface end 44, a feed port 47 and a sealing 53. Between the internal section 51 and the external section there is a shoulder 54.

According to one embodiment the external section 52 comprises an external interface end 43, a fat input conduit 45, a coupling for a harvesting tube 46, an air output conduit 48 and a coupling for a suction tube 49 that are similar to the ones explained above in accordance with FIGS. 1-4.

According to one embodiment the external section 52 comprises a grip surface that is ergonomically shaped. The shape of the grip surface is flattened such that one dimension is smaller than the other, for example from the top view of the connector the grip surface has an oval shape.

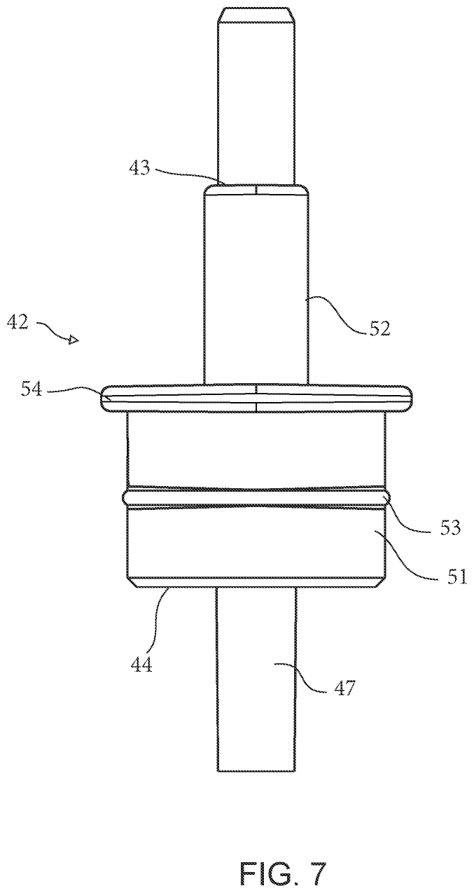

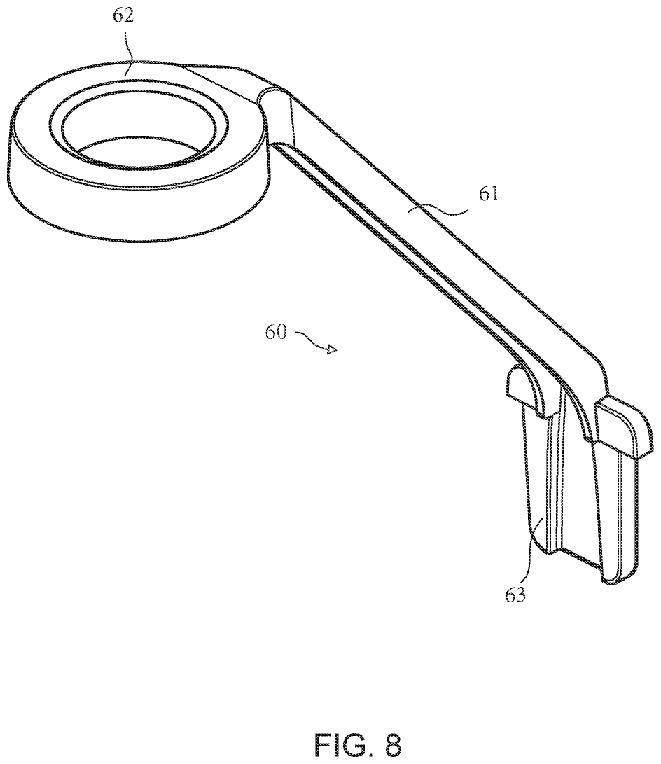

FIG. 7 illustrates side view of a connector in accordance with the same embodiments as FIG. 6.

FIG. 8 illustrates a holder in accordance with at least one embodiment of the current invention. The holder is for supporting the receptive syringe during the fat harvesting process.

According to one embodiment, the holder 60 has a stem 61 with two ends. The first end 62 is configured to hold the receptive syringe and the second end 63 is configured to be connected to the vacuum machine.

According to one embodiment the first end 62 has a hole matching the size of diameter of the outer surface of the receptive syringe.

It is to be understood that the embodiments of the invention disclosed are not limited to the particular structures, process steps, or materials disclosed herein, but are extended to equivalents thereof as would be recognized by those ordinarily skilled in the relevant arts. It should also be understood that terminology employed herein is used for the purpose of describing particular embodiments only and is not intended to be limiting.

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various embodiments and example of the present invention may be referred to herein along with alternatives for the various components thereof. It is understood that such embodiments, examples, and alternatives are not to be construed as de facto equivalents of one another, but are to be considered as separate and autonomous representations of the present invention.

Furthermore, the described features, structures, or characteristics may be combined in any suitable manner in one or more embodiments. In the following description, numerous specific details are provided, such as examples of lengths, widths, shapes, etc., to provide a thorough understanding of embodiments of the invention. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

The verbs “to comprise” and “to include” are used in this document as open limitations that neither exclude nor require the existence of also un-recited features. The features recited in depending claims are mutually freely combinable unless otherwise explicitly stated. Furthermore, it is to be understood that the use of “a” or “an”, i.e. a singular form, throughout this document does not exclude a plurality.

### INDUSTRIAL APPLICABILITY

The present connector for fat harvesting system and method for harvesting fat from a cannula can be used in plastic, dermatological and cosmetic surgery. In particular, it can be used in fat transfer for cosmetic or health reasons.

### REFERENCE SIGNS LIST

1 Connector
2 Body
3 External interface end
4 Internal interface end
5 Fat input conduit
6 Coupling for a harvesting tube
7 Feed port
8 Air output conduit
9 Coupling for a suction tube
10 Suction port
11 Internal section
12 External section
13 Sealing
14 Shoulder
25 First syringe
26 Coupling for a harvesting tube
29 Coupling for a suction tube
30 Suction tube
31 Harvesting tube
32 Second syringe
33 Luerlock connector
42 Body
43 External interface end
44 Internal interface end
45 Fat input conduit
46 Coupling for a harvesting tube
47 Feed port
48 Air output conduit
49 Coupling for a suction tube
51 Internal section
52 External section
53 Sealing
54 Shoulder
60 Holder
61 Stem
62 First end
63 Second end

### CITATION LIST

### Patent Literature

WO 2014098909 A1
U.S. Pat. No. 8,172,832 B1
U.S. Pat. No. 8,858,518 B2

1. A sterile connector for fat harvesting, comprising:
a body configured to be at least partially inserted into a receptive syringe chamber and comprising:
an external interface end for facing the ambient space, and
an internal interface end for insertion into the syringe chamber,
a fat input conduit extending through the body and comprising:
a coupling for a harvesting tube at the external interface end of the body, and
a feed port at the internal interface end of the body for feeding the harvested fat into the syringe chamber, and
an air output conduit extending through the body and comprising:
a coupling for a suction tube at the external interface end of the body, and
a suction port at the internal interface end of the body for providing a partial vacuum into the syringe chamber.

2. The connector according to claim 1, wherein the body comprises:
an internal section housing the internal interface end, wherein the internal section is configured to be inserted into a receptive syringe chamber, and
an external section housing the external interface end, wherein the external section is configured to stay out of the receptive syringe chamber.

3. The connector according to claim 2, wherein the internal section is adapted to form a hermetic seal when inserted into a receptive syringe chamber.

4. The connector according to claim 3, further comprising a sealing to form the hermetic seal.

5.-6. (canceled)

7. The connector according to claim 1, wherein the external section has a grip surface.

8. (canceled)

9. The connector according to claim 2, wherein when inserted into the syringe chamber the fat input conduit extends deeper into the syringe chamber than the air output conduit.

10. The connector according to claim 9, wherein the fat input conduit extends at least 10 mm deeper into the syringe chamber than the air output conduit.

11. The connector according to claim 2, wherein the body has a shoulder for preventing the external interface end from being inserted into the receptive syringe chamber.

12. The connector according to claim 2, wherein the connector is made of a polymer material.

13. (canceled)

14. The connector according to claim 2, wherein the connector is formed of one solid piece.

15. (canceled)

16. The connector according to claim 2, wherein the length of the internal section is at least 1 cm, for example at least 1.7 cm, such as 2-3 cm.

17. The connector according to claim 2, wherein the length of the external section is at least 0.5 cm, for example at least 1.5 cm, such as 1.5-3.0 cm, or at least 20 mm.

18. The connector according to claim 2, wherein the diameter of the internal section is 26.5-27.5 mm, so that it is configured to fit tightly into the syringe chamber of a standard 50 ml syringe.

19. The connector according to claim 2, wherein the diameter of the internal section is 14-15.5 mm, so that it is configured to fit tightly into the syringe chamber of a standard 20 ml syringe.

20. (canceled)

21. The connector according to claim 2, wherein the connector is made of biocompatible polypropylene.

22. (canceled)

23. A fat harvesting system including a connector, a first syringe, a harvesting tube and at least one of a plug and a second syringe with a luerlock connector, wherein:
the connector is hermetically sealed to the first syringe,
the harvesting tube is connected to the coupling for a harvesting tube, and
the plug and or the second syringe with the luerlock connector is connected to the first syringe,
and wherein the connector comprises:
a body configured to be at least partially inserted into a receptive syringe chamber and comprising:
an external interface end for facing the ambient space, and
an internal interface end for insertion into the syringe chamber,
an fat input conduit extending through the body and comprising:
a coupling for a harvesting tube at the external interface end of the body, and
a feed port at the internal interface end of the body for feeding the harvested fat into the syringe chamber, and
an air output conduit extending through the body and comprising:
a coupling for a suction tube at the external interface end of the body, and
a suction port at the internal interface end of the body for providing a partial vacuum into the syringe chamber.

24.-27. (canceled)

28. The fat harvesting system according to claim 23, wherein the first syringe is a standard 50 ml or 20 ml syringe.

29.-30. (canceled)

31. A sterile connector for fat harvesting, comprising:
a body configured to be partially inserted into a receptive syringe chamber and comprising:
an external interface end for facing the ambient space,
an internal interface end for insertion into the syringe chamber, and
a shoulder for preventing the external interface end from being inserted into the receptive syringe chamber,
an air output conduit extending through the body and comprising:
a coupling for a suction tube at the external interface end of the body, and
a suction port at the internal interface end of the body for providing a partial vacuum into the syringe chamber, the suction port configured to be located inside the syringe chamber, and
an fat input conduit extending through the body and comprising:
a coupling for a harvesting tube at the external interface end of the body, and
a feed port at the internal interface end of the body for feeding the harvested fat into the syringe chamber, the feed port configured to extend deeper into the syringe chamber than the suction port.

32. The sterile connector according to claim 31, wherein the feed port is configured to extend at least 10 mm deeper into the syringe chamber than the suction port.

33. The sterile connector according to claim 31, further comprising a sealing configured to form a hermetic seal when inserted into a receptive syringe chamber.
